(19) United States
(12) Patent Application Publication
(10) Pub. No.: US 2021/0025863 A1
(43) Pub. Date: Jan. 28, 2021

(54) FATIGUE LEVEL ESTIMATION METHOD AND CREATING METHOD FOR DATABASE FOR FATIGUE LEVEL ESTIMATION

(71) Applicant: JTEKT CORPORATION, Osaka (JP)

(72) Inventors: Yousuke NAGANO, Yao-shi (JP); Kenichi ISEDA, Fujiidera-shi (JP)

(73) Assignee: JTEKT CORPORATION, Osaka (JP)

(21) Appl. No.: 16/919,445

(22) Filed: Jul. 2, 2020

(30) Foreign Application Priority Data
Jul. 22, 2019 (JP) 2019-134440

(51) Int. Cl. G01N 33/204; G01N 23/207; G06F 16/23

(52) U.S. Cl. CPC G01N 33/204; G01N 23/207; G01N 2223/0565; G01N 2223/607; G06F 16/2379

(57) ABSTRACT

A fatigue level estimation method includes estimating a fatigue portion in a metal material, measuring a distribution of a misorientation in the fatigue portion, obtaining a specific area ratio of the fatigue portion based on the distribution of the misorientation in the fatigue portion, and obtaining an estimated fatigue level of the metal material based on at least one of the specific area ratio of the fatigue portion and a degree of change in the specific area ratio of the fatigue portion. The specific area ratio of the fatigue portion is a ratio of a specific area existing in a measurement area of the fatigue portion.

### CROSS-REFERENCE TO RELATED APPLICATION

This application claims priority to Japanese Patent Application No. 2019-134440 filed on Jul. 22, 2019, incorporated herein by reference in its entirety.

### BACKGROUND

### 1. Technical Field

The disclosure relates to a fatigue level estimation method for estimating a fatigue level of a metal material, and a creation method for a database for a fatigue level estimation, which is used in the fatigue level estimation method.

### 2. Description of Related Art

As a method for estimating the fatigue level of metal parts, the amount of strain generated in crystal grains of a metal is quantitatively measured by X-ray diffraction, and the fatigue level is estimated based on the amount of the strain (for example, see Japanese Unexamined Patent Application Publication No. 11-344454 (JP 11-344454 A)).

### SUMMARY

The measurement of the amount of strain using the X-ray diffraction has an advantage that the measurement can be performed without destructing the metal material. On the other hand, since a penetration depth of X-rays when irradiation is performed from the outside is several μm, it is difficult for the X-rays to reach a region where fatigue has occurred in cases of fatigue that has occurred in a region relatively inward from a surface, such as rolling fatigue that has occurred in bearing parts, etc. Thus, there are cases in which it is difficult to perform accurate measurement of the amount of strain. Accordingly, the region relatively inward from the surface of the metal material may be measured using the X-ray diffraction by cutting the metal material and irradiating a cross section of the metal material with the X-rays.

Here, in irradiating the cross section of the metal material with the X-rays, to desirably increase the accuracy of a measurement result to improve an estimation accuracy of the fatigue level, it is necessary to increase a diffraction intensity by expanding an irradiation range of the X-rays or by extending an irradiation time of the X-rays. However, the expansion of the irradiation range increases the possibility that the X-rays is irradiated to portions other than a fatigue portion. The measurement result may thus include information on portions other than the fatigue portion, which causes an issue of a deteriorated accuracy of the measurement result. Further, the extension of the irradiation time increases a measurement time and load on an X-ray diffractometer, which also causes a cost issue.

As described above, in estimating the fatigue level based on the cross section of the metal material using the X-ray diffraction, to desirably increase the diffraction intensity to increase the accuracy of a measurement value, on the contrary, the accuracy of the measurement value may be decreased or the cost may be increased. Thus, there is an issue that it is difficult to further increase the estimation accuracy of the fatigue level.

The disclosure provides a technique that can further increase the estimation accuracy of the fatigue level of the metal material.

In estimating the fatigue level of the metal material, the inventors of the present application focused on measurement of local misorientation using electron backscatter diffraction (EBSD) that can quantify a state of strain in crystal grains generated in the metal material, as in the case of the X-ray diffraction method. It was found that, when a distribution of the local misorientation was measured in a predetermined range of the metal material by EBSD, there was a good correlation between an existence ratio of an area in which the local misorientation was a predetermined set value in the measurement area and the fatigue level of the metal material. The inventors of the present application completed the disclosure based on this finding.

A fatigue level estimation method for estimating a fatigue level of a metal material, the fatigue level estimation method according to a first aspect of the disclosure includes estimating a fatigue portion in which fatigue has occurred in the metal material, measuring a distribution of a misorientation in the fatigue portion, obtaining a specific area ratio of the fatigue portion based on the distribution of the misorientation in the fatigue portion, and obtaining an estimated fatigue level of the metal material based on at least one of the specific area ratio of the fatigue portion and a degree of change in the specific area ratio of the fatigue portion. The specific area ratio of the fatigue portion is a ratio of a specific area existing in a measurement area of the fatigue portion, and the specific area is an area in which the misorientation is a predetermined set value.

According to the fatigue level estimation method of the above aspect, since the misorientation measured using the EBSD is used to obtain the estimated fatigue level, it is possible to perform measurement in a smaller range as compared with the X-ray diffraction method. As a result, the measurement area can be accurately aligned to the fatigue portion in which fatigue has occurred, and the distribution of the misorientation that is the measurement result of the fatigue portion can be obtained accurately. Further, the specific area ratio of the fatigue portion and the degree of change in the specific area ratio of the fatigue portion each has a correlation with the estimated fatigue level of the metal material, which allows the estimated fatigue level of the metal material to be obtained.

As described above, according to the fatigue level estimation method of the above configuration, the estimated fatigue level can be obtained while accurately obtaining the measurement result of the fatigue portion, and the estimation accuracy of the fatigue level can be further increased.

The fatigue level estimation method according to the above aspect may further include measuring a distribution of a misorientation in a surface layer portion between a rolling surface of the metal material and the fatigue portion, and determining a specific area ratio of the surface layer portion based on the distribution of the misorientation in the surface layer portion. The fatigue portion is a portion in which rolling fatigue has occurred and exists in a range in which a depth distance from the rolling surface of the metal material is a predetermined value or more. The specific area ratio of the surface layer portion is a ratio of the specific area existing in a measurement area of the surface layer portion. Obtaining the estimated fatigue level of the metal material may be obtaining the degree of change in the specific area ratio of the fatigue portion based on the specific area ratio of the fatigue portion and the specific area ratio of the surface layer portion.

When fatigue that has occurred in the metal material is rolling fatigue, the surface layer portion between the rolling surface and the fatigue portion maintains its state before the rolling fatigue has occurred. Thus, it can be considered that the ratio of the specific area existing in the predetermined range in the surface layer portion is substantially the same as the initial value of the existence ratio of the specific area before fatigue has occurred in the fatigue portion. Accordingly, an increment that has increased from the initial value to the current timing in the specific area ratio of the fatigue portion can be obtained based on the specific area ratio of the fatigue portion and the specific area ratio of the surface layer portion and thereby the degree of change in the specific area ratio of the fatigue portion can be obtained.

In the fatigue level estimation method according to the above aspect, the misorientation may be one of a kernel average misorientation (KAM) value, a local average misorientation (LAM) value, and a local orientation spread (LOS) value. In this case, the estimated fatigue level of the metal material can be preferably obtained using any of the values.

In obtaining the estimated fatigue level of the metal material, when the estimated fatigue level of the metal material is obtained based on the specific area ratio of the fatigue portion, the predetermined set value may be set within a range of 2 degrees to 8 degrees. When the predetermined set value is 9 deg or more, the increment of the specific area ratio of the fatigue portion with respect to the increment of the estimated fatigue level may be small, or a range in which the relationship between the specific area ratio of the fatigue portion and the estimated fatigue level does not show a monotonic increase may appear. Accordingly, the accuracy of the estimated fatigue level may be decreased. Therefore, the predetermined set value is preferably set in the range of 2 deg to 8 deg.

The predetermined set value may be set within a range of 3 degrees to 5 degrees. When the predetermined set value is smaller than 3 deg, the relationship between the specific area ratio of the fatigue portion and the fatigue level may not always show a linear relationship, and the accuracy of the estimated fatigue level that is obtained may be slightly decreased. Similarly, when the set value is larger than 5 deg, the relationship between the specific area ratio of the fatigue portion and the fatigue level may not always show the linear relationship, and the accuracy of the estimated fatigue level that is obtained may be slightly decreased. By setting the set value within the range of 3 deg to 5 deg, the correlation between the specific area ratio of the fatigue portion and the fatigue level can show a good linear relationship.

In obtaining the estimated fatigue level of the metal material, when the estimated fatigue level of the metal material is obtained based on the degree of change in the specific area ratio of the fatigue portion, the predetermined set value may be set within a range of 2 degrees to 9 degrees. This is because a good correlation between the degree of change and the estimated fatigue level can be obtained in the above case. When the predetermined set value is 10 deg or more, an abnormal value may appear or a range in which the relationship between the degree of change and the estimated fatigue level does not show a monotonic increase may appear. Accordingly, the accuracy of the estimated fatigue level may be decreased. Therefore, the predetermined set value is preferably set in the range of 2 deg to 9 deg.

The fatigue level estimation method according to the above aspect may further include creating a database showing a relationship between the fatigue level of the metal material and at least one of the specific area ratio of the fatigue portion and the degree of change in the specific area ratio of the fatigue portion. In obtaining the estimated fatigue level of the metal material, the estimated fatigue level of the metal material may be obtained by referring to the database.

A creation method for a database for a fatigue level estimation for creating the database for the fatigue level estimation of a metal material, the database being used in obtaining the estimated fatigue level of the metal material based on at least one of the specific area ratio of the fatigue portion and the degree of change in the specific area ratio of the fatigue portion in the fatigue level estimation method according to claim 1, the creation method for the database according to a second aspect of the disclosure includes obtaining a plurality of test samples made of the same material as the metal material, each of the test samples having a different fatigue level, estimating the fatigue portion in which fatigue has occurred in each of the test samples, measuring a distribution of a misorientation in the fatigue portion in each of the test samples, obtaining the specific area ratio of the fatigue portion for each of the test samples based on the distribution of the misorientation in the fatigue portion, and associating the fatigue level of each of the test samples with at least one of the specific area ratio of the fatigue portion of each of the test samples and the degree of change in the specific area ratio of the fatigue portion of each of the test samples to create the database for the fatigue level estimation.

According to the above aspect, it is possible to obtain a database for fatigue level estimation that can further increase the estimation accuracy of the fatigue level.

According to the above aspects, it is possible to further increase the estimation accuracy of the fatigue level of the metal material.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram for describing a method for obtaining a kernel average misorientation (KAM) value;

FIG. 2 is a flowchart showing a fatigue level estimation method for a metal material according to an embodiment;

FIG. 3 is a diagram for describing steps from step Si to step S3 in FIG. 2;

FIG. 4 shows an example of an association table in which the KAM values (classes) and existence ratios (relative frequencies) are associated with each other, which are obtained from a distribution of the KAM values;

FIG. 5A is an example of a histogram indicated based on the association table shown in FIG. 4;

FIG. 5B is an example of a histogram indicated based on the association table shown in FIG. 4;

FIG. 6 is a diagram showing a fatigue level database in a graph;

FIG. 7 is a block diagram showing an example of a calculation device for obtaining an estimated fatigue level;

FIG. 8A is a graph showing a relationship between a specific area ratio of a surface layer portion and a calculated life ratio obtained when creating the fatigue level database shown in FIG. 6;

FIG. 8B is a graph showing a relationship between a specific area ratio of a fatigue portion and the calculated life ratio obtained when creating the fatigue level database shown in FIG. 6;

FIG. 9A is a graph of the fatigue level database showing a relationship between a degree of change in the specific area ratio of the fatigue portion and the calculated life ratio when a set value is set in a range of 2 deg to 4 deg;

FIG. 9B is a graph of the fatigue level database showing the relationship between the degree of change in the specific area ratio of the fatigue portion and the calculated life ratio when the set value is set in a range of 5 deg to 7 deg;

FIG. 9C is a graph of the fatigue level database showing the relationship between the degree of change in the specific area ratio of the fatigue portion and the calculated life ratio when the set value is set in a range of 8 deg to 10 deg;

FIG. 9D is a graph of the fatigue level database showing the relationship between the degree of change in the specific area ratio of the fatigue portion and the calculated life ratio when the set value is set in a range of 11 deg to 13 deg;

FIG. 10A is a graph of the fatigue level database showing the relationship between the specific area ratio of the fatigue portion and the calculated life ratio when the set value is set in a range of 2 deg to 7 deg; and

FIG. 10B is a graph of the fatigue level database showing the relationship between the specific area ratio of the fatigue portion and the calculated life ratio when the set value is set in a range of 8 deg to 13 deg.

### DETAILED DESCRIPTION OF EMBODIMENTS

Next, a preferred embodiment of the disclosure will be described with reference to the accompanying drawings.

### Measurement of Local Misorientation Average Value

In the present embodiment, a local misorientation average value of metal, which is represented by a kernel average misorientation (KAM) value, is used to estimate a fatigue level of a metal material. The KAM value indicates an average value of a local crystal misorientation of the metal. A crystal orientation is obtained from a diffraction pattern of backscattered electrons detected by an electron backscatter diffraction (EBSD) detector provided in a scanning electron microscope (SEM), and the KAM value is obtained based on the obtained crystal orientation.

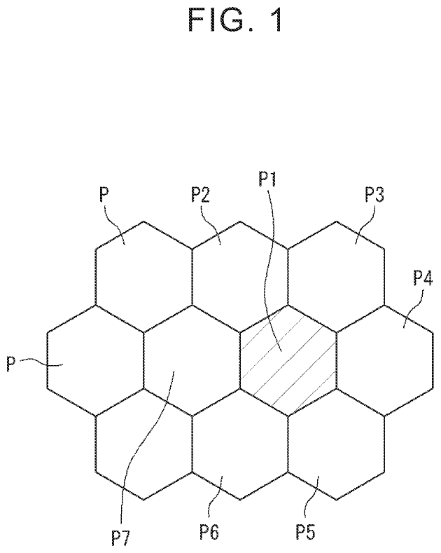

FIG. 1 is a diagram for describing a method for obtaining the KAM value. To obtain the KAM value, for example, a plurality of virtual polygonal (hexagonal in an example in FIG. 1) measurement point areas P is set in a measurement area of the metal material. The measurement point areas P are measurement points for the KAM value. The measurement area is scanned by an electron beam at a pitch determined by the sizes of the measurement point areas P. The KAM value is obtained for each of the measurement point areas P. Accordingly, the measurement point areas P constitute a unit for indicating the distribution of the KAM values described later.

In FIG. 1, to obtain the KAM value of a hatched measurement point area P1, first, the crystal misorientation between the measurement point area P1 and the measurement point areas P2, P3, P4, P5, P6, and P7 surrounding the measurement point area P1 are obtained. That is, the crystal misorientation between the measurement point area P1 and the measurement point area P2, the crystal misorientation between the measurement point area P1 and the measurement point area P3, the crystal misorientation between the measurement point area P1 and the measurement point area P4, the crystal misorientation between the measurement point area P1 and the measurement point area P5, the crystal misorientation between the measurement point area P1 and the measurement point area P6, and the crystal misorientation between the measurement point area P1 and the measurement point area P7 are obtained. Then, the calculated six crystal misorientations are averaged. The value thereby obtained is the KAM value of the measurement point area P1.

The crystal misorientation in crystal grains is caused by strain generated in the crystal grains and does not show a large value. When the crystal misorientation shows a relatively large value, it is considered that the value is affected by a crystal grain boundary or another adjacent crystal. In the present embodiment, the measurement point area P of which the average value (KAM value) of the crystal misorientation described above is 15 degrees (hereinafter also referred to as “deg”) or more is defined as a grain boundary, and only data of the grains having the KAM values within 0 deg to 15 deg is used for a database for fatigue level estimation (described later).

In the present embodiment, the distribution of the KAM values in the measurement area set for the metal material is measured, and a ratio of a specific area existing in the measurement area is obtained based on the distribution of the measured KAM values. A fatigue level of the metal material is estimated using the ratio of the specific area. The specific area is an area within the measurement area and the KAM value in the specific area is a predetermined value set in advance. The specific area will be described in detail later.

### Fatigue Level Estimation of Metal Material

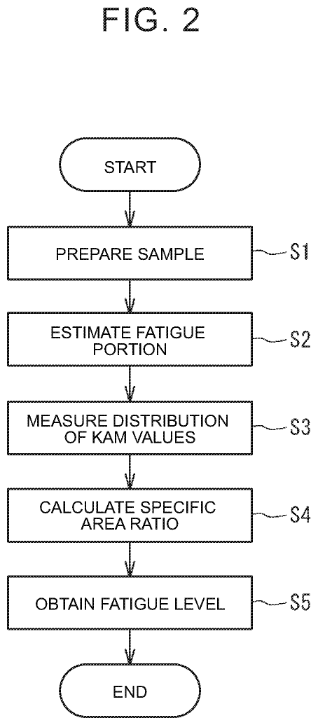

FIG. 2 is a flowchart showing a fatigue level estimation method for the metal material according to the present embodiment. In the present embodiment, the distribution of the KAM values in a portion in which fatigue has occurred in the metal material (fatigue portion) is measured. In this method, first, as a preparatory step for estimating the fatigue level, a sample of the metal material that is a sample for estimating the fatigue level is prepared (step S1). Next, the prepared metal material is used to estimate the fatigue portion in which fatigue has occurred in the metal material (step S2). Then, the distribution of the KAM values in the estimated fatigue portion is measured (step S3). Subsequently, based on the distribution of the KAM values in the fatigue portion measured in step S3, the ratio of the specific area existing in the measurement area of the KAM values is calculated (step S4). Lastly, based on the ratio of the specific area obtained in step S4, a degree of change in the ratio of the specific area in the fatigue portion is obtained, and an estimated fatigue level of the metal material is obtained based on the degree of change (step S5).

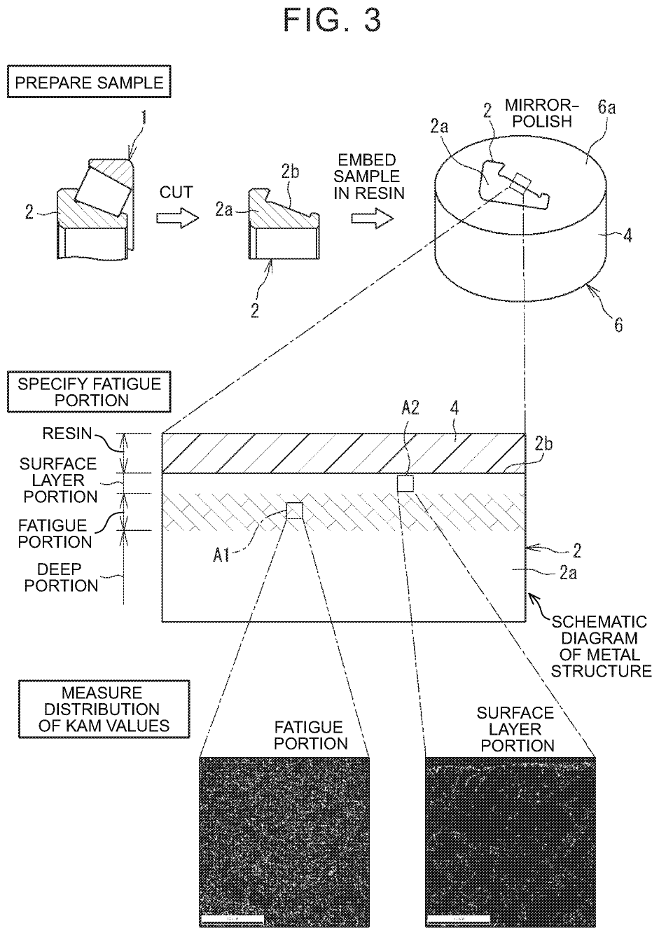

FIG. 3 is a diagram for describing the steps from step Si to step S3 in FIG. 2. The following description describes a case in which an inner ring of a tapered roller bearing is used as the metal material of which the fatigue level is estimated. In FIG. 3, in preparing the sample (step Si in FIG. 2), an inner ring 2 of a tapered roller bearing 1 is cut by a high-speed cutter or the like, and the cut inner ring 2 is embedded in a resin 4 to obtain a measurement sample 6. The inner ring 2 is embedded in the resin 4 such that a cross section 2a of the inner ring 2 is exposed on an observation surface 6a of the measurement sample 6. The observation surface 6a on which the cross section 2a is exposed is mirror-polished. The cross section 2a is thus mirror-polished. The inner ring 2 is made of, for example, alloy steel for machine structure or carbon steel for machine structure, and is heat-treated in advance.

Next, in FIG. 3, in estimating the fatigue portion (step S2 in FIG. 2), the mirror-polished cross section 2a of the inner ring 2 is etched with a predetermined corrosive solution, and a metal structure of the cross section 2a is observed with a metallographic microscope or the like to estimate the fatigue portion based on the observation result of the metal structure. FIG. 3 schematically shows the metal structure of the cross section 2a of the inner ring 2 of the tapered roller bearing 1, which is observed on the observation surface 6a. The schematic diagram of the metal structure in FIG. 3 shows the resin 4 and the cross section 2a of the inner ring 2 embedded in the resin 4.

For example, when the metal structure has a region in which refinement of the crystal grains is remarkably advanced as compared with its surroundings, it can be estimated that fatigue has occurred in that region. In a bearing ring of the rolling bearing, rolling fatigue occurs in a region further inward of a raceway surface (rolling surface) on which the rolling elements roll rather than in a region near the raceway surface. In the schematic diagram of the metal structure in FIG. 3, the inner ring 2 has a region of the fatigue portion in a region further inward of the raceway surface 2b. The region of the fatigue portion is estimated by the fact that the refinement of the crystal grains is advanced than other regions. The region of the fatigue portion is generally generated in a depth distance of 50 μm to 200 μm from the raceway surface 2b. In a surface layer portion located between the raceway surface 2b and the region of the fatigue portion and in a deep portion located inward of the fatigue portion, the refinement of the crystal grains is not advanced like the fatigue portion and there are no signs of fatigue.

Thus, the fatigue portion that is a portion in which fatigue is advanced compared to its surroundings is estimated by observing the metal structure. For example, positional information of the fatigue portion such as a distance between the raceway surface 2b and the fatigue portion and a width of the fatigue portion in a depth direction is obtained by observing the structure. Note that when the metal material is used under steady conditions with a pre-known load, the depth and position of the fatigue portion may be estimated by calculation.

Next, in FIG. 3, in measuring the distribution of the KAM values (step S3 in FIG. 2), the measurement sample 6 of which the metal structure has been observed is mirror-polished again, and the measurement sample 6 that has been mirror-polished again is set on the SEM to measure the distribution of the KAM values by the EBSD. To measure the distribution of the KAM values, first, the measurement area is set. When measuring the distribution of the KAM values in the fatigue portion, a measurement area A1 (schematic diagram of the metal structure in FIG. 3) is set in the region of the fatigue portion. The distribution of the KAM values in the measurement area A1 is measured by the EBSD.

As described above, the distribution of the KAM values is measured in units of the measurement point area P shown in FIG. 1. Accordingly, the electron beam is scanned at a pitch determined by the size of the measurement point area P, and the KAM value is measured for each measurement point area P included in the measurement area A1. Thereby, the distribution of the KAM values in the measurement area A1 is measured.

In the present embodiment, in addition to the distribution of the KAM values in the fatigue portion, the distribution of the KAM values in the surface layer portion is also measured. Similar to the distribution measurement of the KAM values in the fatigue portion, the distribution measurement of the KAM values in the surface layer portion is performed by setting a measurement area A2 (schematic diagram of the metal structure in FIG. 3) in a region of the surface layer portion and measuring the distribution of the KAM values in the measurement area A2. The measurement areas A1, A2 are, for example, set in a square shape with one side of 30 μm.

The distribution of the KAM values in the fatigue portion obtained in step S3 is obtained as a plurality of KAM values associated with each measurement point area P included in the measurement area A1. That is, the distribution of the KAM values in the fatigue portion is a data group in which the positional information of each measurement point area P in the measurement area A1 and the KAM value corresponding thereto are associated with each other. Similarly, the distribution of the KAM values in the surface layer portion is a data group in which the positional information of each measurement point area P in the measurement area A2 and the KAM value corresponding thereto are associated with each other.

Note that the distribution of the KAM values may include a KAM value in a complementary point that complements adjacent measurement point areas P. The KAM value in the complementary point is, for example, a value calculated by a calculation device for obtaining the distribution of the KAM values. The calculation device can set the complementary point and calculate the KAM value in the set complementary point based on the surrounding KAM values. When using the KAM value of the complementary point, the complementary point is treated as the measurement point area P.

In FIG. 3, as an example of the measurement result, the distribution of the KAM values is shown as a map. The distribution of the KAM values is the data group in which the positional information of each measurement point area P in the measurement area A1 (A2) and the KAM value corresponding thereto are associated with each other. Thus, by setting a contrast according to the magnitude of the KAM values, the distribution of the KAM values can be represented as a map.

Next, as shown in step S4 in FIG. 2, based on the distribution of the KAM values, the ratio of the specific area (specific area ratio) existing in the measurement area when the KAM values are measured is obtained. To obtain the specific area ratio, first, an existence ratio (existence probability) of each KAM value is obtained from the obtained distribution of the KAM values. As described above, the distribution of the KAM values includes the KAM values associated with each measurement point area P included in the measurement area A1 (A2). Thus, the distribution of the KAM values can be represented by a frequency distribution in which the KAM values serve as classes and the number of the measurement point areas P serves as the frequency. When the distribution of the KAM values is represented by the frequency distribution, the relative frequency indicates the existence ratio of each class (each KAM value). In this way, the existence ratio of each KAM value in the measurement area A1 (A2) can be obtained from the distribution of the KAM values.

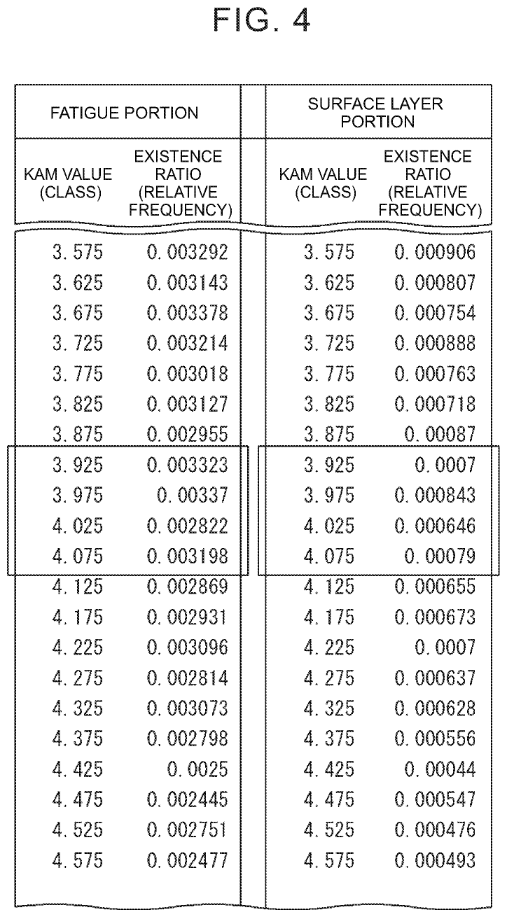

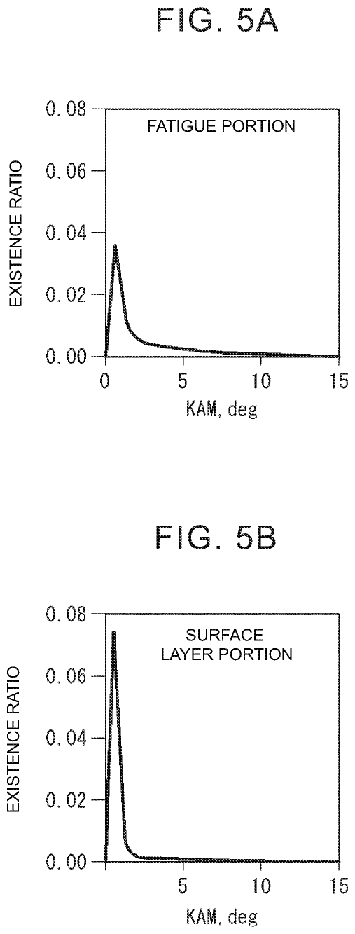

FIG. 4 shows an example of an association table in which the KAM values (classes) and the existence ratios (relative frequencies) are associated with each other, which are obtained from the distribution of the KAM values, and FIG. 5A and FIG. 5B are examples of a histogram indicated based on the association table shown in FIG. 4. In FIG. 5A and FIG. 5B, a horizontal axis indicates the class showing the KAM value, and a vertical axis indicates the existence ratio. The class is set in a range of 0 deg to 15 deg in increments of a preset value (for example, 0.005 deg). The KAM value measured in each measurement point area P is associated with one of the plurality of classes according to a predetermined rule, and is counted as a frequency. As described above, in the present embodiment, when the crystal misorientation is 15 deg or more, the measurement point area P is determined to be the grain boundary. Thus, the KAM values in the range of 0 deg to 15 deg is set as the target of the data to be accumulated. Therefore, the classes in the association table shown in FIG. 4 and the histogram shown in FIG. 5A and FIG. 5B are set in the range of 0 deg to 15 deg.

As described above, the existence ratio of each KAM value in the fatigue portion is obtained from the distribution of the KAM values in the fatigue portion. Further, the existence ratio of each KAM value in the surface layer portion is obtained from the distribution of the KAM values in the surface layer portion.

As described above, after obtaining the existence ratio of each KAM value in the fatigue portion and the existence ratio of each KAM value in the surface layer portion, the specific area ratio is obtained based on the existence ratio of each KAM value. As described above, the specific area is an area existing in the measurement areas A1, A2, and is an area in which the KAM value is a predetermined value set in advance. The specific area ratio can be obtained by referring to the association table showing the existence ratio of each KAM value shown in FIG. 4. For example, when the set value is set to 4 deg, the specific area is an area having the KAM value of 4 deg, and the ratio of the area having the KAM value of 4 deg in the measurement areas A1, A2 is obtained.

In this case, referring to the association table shown in FIG. 4, the existence ratios corresponding to the plurality of (four in the example in FIG. 4) classes having the KAM values in a range from 3.9 deg to 4.1 deg are obtained, and the average value of the existence ratios is obtained. The obtained average value is used as the specific area ratio. As described above, in the present embodiment, the existence ratios corresponding to the KAM values within a predetermined numerical range centered on the set value is obtained, and the average value of the obtained existence ratios is set as the specific area ratio.

In FIG. 4, the four classes surrounded by a square frame in the table are the classes included in the range of the KAM values from 3.9 deg to 4.1 deg. Thus, in FIG. 4, the specific area ratio in the fatigue portion is the average value of 0.0032 of the existence ratios of 0.003323, 0.00337, 0.002822, and 0.003198 that correspond to the four classes within the square frame. Similarly, the specific area ratio in the surface layer portion is the average value of 0.0007 of the existence ratios of 0.0007, 0.000843, 0.000646, and 0.00079.

As described above, the specific area ratio of the fatigue portion, which is the ratio of the specific area existing in the measurement area A1 in the fatigue portion is obtained, and the specific area ratio of the surface layer portion, which is the ratio of the specific area existing in the measurement area A2 in the surface layer portion is also obtained.

Next, as shown in step S5 in FIG. 2, the degree of change in the specific area ratio of the fatigue portion is obtained, and the fatigue level of the inner ring 2 is obtained based on the degree of change. The degree of change in the specific area ratio of the fatigue portion is calculated based on the following equation.

Degree of change in specific area ratio of fatigue portion=((Specific area ratio in fatigue portion)−(initial value))/(initial value)

In the above equation, the initial value is a specific area ratio before fatigue has occurred. The initial value can be obtained, for example, by cutting, before use, the inner ring 2 that has undergone the same manufacturing process as the inner ring 2 of which the fatigue level is to be estimated and measuring the initial value in advance. However, it is difficult to obtain the initial value of the inner ring 2 of a product that has been collected from the field. Thus, in the present embodiment, the specific area ratio of the surface layer portion is used instead of the initial value. As described above, in the bearing ring of the rolling bearing, the rolling fatigue occurs in the region further inward of the raceway surface on which the rolling elements roll rather than in the region near the raceway surface, and no significant sign of fatigue is found in the surface layer portion. Thus, it can be considered that the specific area ratio of the surface layer portion is substantially the same as the specific area ratio of the fatigue area before fatigue has occurred. Accordingly, in the present embodiment, the degree of change in the specific area ratio of the fatigue portion is calculated by using the specific area ratio of the surface layer portion instead of the initial value. This makes it possible to obtain the initial value even with a product that has been collected from the field, and it is possible to accurately obtain the degree of change in the specific area ratio of the fatigue portion.

As described above, when the specific area ratio of the fatigue portion is 0.0032 and the specific area ratio of the surface layer portion is 0.0007, the degree of change in the specific area ratio of the fatigue portion is 3.57. In this way, the degree of change in the specific area ratio of the fatigue portion is obtained based on the specific area ratio of the fatigue portion and the specific area ratio of the surface layer portion.

Next, the estimated fatigue level of the inner ring 2 is obtained based on the degree of change in the specific area ratio of the fatigue portion. The estimated fatigue level of the inner ring 2 is obtained by referring to a fatigue level database created in advance.

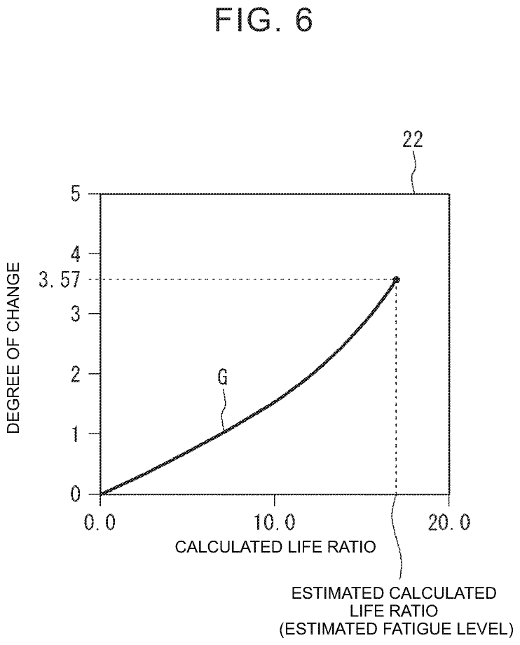

FIG. 6 shows the fatigue level database in a graph. A fatigue level database 22 is data (numerical values or mathematical equations) showing a relationship between the degree of change in the specific area ratio of the fatigue portion and the fatigue level of the inner ring 2. A creation method for the fatigue level database 22 will be described later.

The fatigue level database 22 shown in FIG. 6 shows the relationship between the degree of change in the specific area ratio of the fatigue portion when the set value is 4 deg and the fatigue level. The fatigue level database 22 uses a calculated life ratio as the fatigue level. The calculated life ratio is a ratio of an actual life of the tapered roller bearing 1 with respect to a basic rating life L10 of the tapered roller bearing 1. The basic rating life L10 is a rating life when a reliability is 90% under normal use conditions. The term “life” as used herein refers to the total number of rotations of one bearing ring with respect to another bearing ring until the first sign of material fatigue appears on either of the bearing ring or the rolling elements of the bearing. Further, the term “reliability” refers to the ratio of the number of the bearings expected to reach or exceed a specified life with respect to the total number of the bearings when a group of the same bearings are operated under the same conditions. The term “rating life” refers to a predicted value of a life based on a basic dynamic radial load rating or a basic dynamic axial load rating.

As shown in the fatigue level database 22 in FIG. 6, the degree of change in the specific area ratio of the fatigue portion has a correlation with the calculated life ratio (fatigue level). More specifically, the degree of change in the specific area ratio of the fatigue portion and the calculated life ratio have a substantially linear relationship. The fatigue level database 22 is stored in, for example, the calculation device for obtaining the estimated fatigue level. FIG. 7 is a block diagram showing an example of the calculation device used for obtaining the estimated fatigue level. A calculation device 10 is constituted of a computer including a processing unit 12 including a central processing unit (CPU) and the like, a storage unit 14 including a hard disk, a memory, etc., and an input/output interface 16. The storage unit 14 stores a program necessary for operating the calculation device 10 and various data. The function of the calculation device 10 is achieved by the processing unit 12 that executes the program stored in the storage unit 14. The processing unit 12 may achieve the function of the calculation device 10 by reading the program stored in a computer-readable storage medium.

The input/output interface 16 is connected to an input device 18 such as a keyboard and a mouse and an output device 20 such as a display and a printer. The input/output interface 16 inputs and outputs various pieces of information via the input device 18 and the output device 20.

As shown in FIG. 7, the fatigue level database 22 is stored in the storage unit 14. When the degree of change in the specific area ratio of the fatigue portion is provided to the calculation device 10 through the input device 18, the processing unit 12 refers to the fatigue level database 22 and obtains the calculated life ratio corresponding to the provided degree of change.

For example, when a value of 3.57 is provided to the calculation device 10 as the degree of change in the specific area ratio of the fatigue portion, the processing unit 12 refers to the fatigue level database 22 (FIG. 6) and obtains the calculated life ratio corresponding to the value of the degree of change of 3.57. In FIG. 6, the processing unit 12 identifies a point M in which the value of the degree of change is 3.57 in a line G, and obtains the calculated life ratio corresponding to the point M as the estimated fatigue level. In the case of FIG. 6, the calculated life ratio corresponding to the value of the degree of change of 3.57 is about 17. Thus, the processing unit 12 outputs 17 as the estimated calculated life ratio, which serves as the estimated fatigue level of the inner ring 2, through the output device 20.

As described above, in the present embodiment, since the KAM values measured by the EBSD is used to obtain the estimated fatigue level, it is possible to perform measurement in a smaller range as compared with the X-ray diffraction method. Thus, the measurement area can be accurately aligned to the fatigue portion in which fatigue has occurred, and the distribution of the KAM values, which is the measurement result of the fatigue portion, can be obtained accurately. Further, the degree of change in the specific area ratio of the fatigue portion, which is obtained based on the distribution of the KAM values, has a correlation with the calculated life ratio (fatigue level) of the tapered roller bearing 1 that is a metal material, allowing the estimated calculated life ratio to be obtained. As described above, according to the above configuration, the estimated fatigue level can be obtained while accurately obtaining the measurement result of the fatigue portion, and the estimation accuracy of the fatigue level can be further increased.

Further, in the present embodiment, since the estimated fatigue level is obtained using the degree of change in the specific area ratio of the fatigue portion, it is possible to suppress an effect of a difference in the initial value of the specific area ratio before fatigue has occurred, and it is thus possible to further increase the estimation accuracy.

The present embodiment exemplifies a case in which the specific area ratio of the fatigue portion (the specific area ratio of the surface layer portion) is obtained based on a single measurement area A1 (A2) to obtain the estimated calculated life ratio. Alternatively, it is possible to obtain a plurality of the calculated life ratios based on a plurality of the measurement areas A1 (A2) to obtain an estimated calculated life ratio from an average value thereof.

### Creation of Fatigue Level Database

Next, the creation method for the fatigue level database 22 will be described. To create the fatigue level database 22, first, a plurality of the tapered roller bearings 1 are prepared, and a durability test is performed on the tapered roller bearings 1 using a durability tester. At this time, the durability test is performed so that the calculated life ratios of the tapered roller bearings 1 are dispersed, for example, in a range of about 0 to 17. Accordingly, the tapered roller bearings 1 having different calculated life ratios (fatigue levels) can be obtained.

Next, the steps from step S1 to step S4 in FIG. 2 are performed for each of the tapered roller bearings 1 having different calculated life ratios. This makes it possible to obtain the specific area ratio of the fatigue portion, the specific area ratio of the surface layer portion, and the degree of change in the specific area ratio of the fatigue portion for each of the inner rings 2 of the tapered roller bearings 1.

Here, when obtaining the specific area ratio of the fatigue portion and the specific area ratio of the surface layer portion for each of the tapered roller bearings 1, the set value can be set as desired. Thus, it is possible to obtain the specific area ratio of the fatigue portion, the specific area ratio of the surface layer portion, and the degree of change in the specific area ratio of the fatigue portion in the case of the desired set value.

Next, the calculated life ratio of each of the tapered roller bearings 1 is associated with the degree of change in the specific area ratio of the fatigue portion of each of the inner rings 2 of the tapered roller bearings 1 to obtain the fatigue level database 22. When the set value is set to 4 deg, the fatigue level database 22 shown in FIG. 6 is obtained. The fatigue level database 22 may be numerical data in which the calculated life ratio of each of the tapered roller bearings 1 is associated with the degree of change in the specific area ratio of the fatigue portion of each of the inner rings 2 of the tapered roller bearings 1, or may be an approximate equation that can be obtained based on the numerical data.

Similar to the degree of change in the specific area ratio of the fatigue portion, the specific area ratio of the fatigue portion and the specific area ratio of the surface layer portion can also be associated with the calculated life ratio of each of the tapered roller bearings 1.

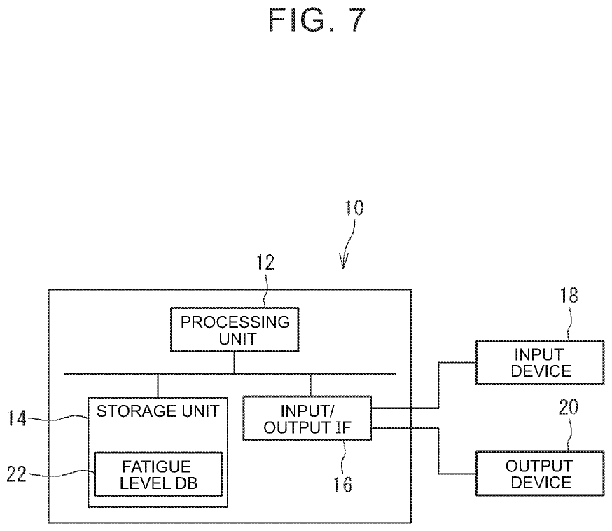

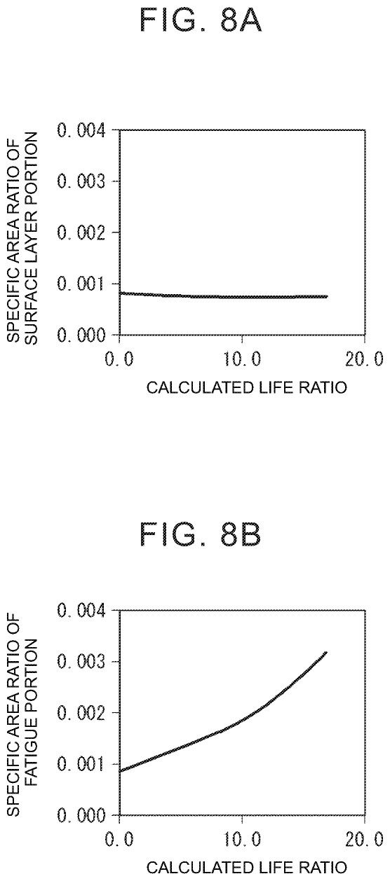

FIG. 8A is a graph showing a relationship between the specific area ratio of the surface layer portion and the calculated life ratio obtained when creating the fatigue level database 22 shown in FIG. 6, and FIG. 8B is a graph showing a relationship between the specific area ratio of the fatigue portion and the calculated life ratio obtained when creating the fatigue level database 22 shown in FIG. 6.

It can be understood from FIG. 8A that the specific area ratio of the surface layer portion hardly changes even when the calculated life ratio that is the fatigue level increases. The inner ring 2 has some distortion due to quenching or the like performed at a manufacturing stage. Even when the inner ring 2 is used, the specific area ratio of the surface layer portion mostly maintains its initial value, indicating that fatigue is hardly found. In other words, FIG. 8A proves that the specific area ratio of the surface layer portion can be considered to have substantially the same value as the specific area ratio before fatigue has occurred.

In the present embodiment, as described above, the specific area ratio of the surface layer portion is used as the initial value (specific area ratio before fatigue has occurred) that is used when obtaining the degree of change in the specific area ratio of the fatigue portion. FIG. 8A indicates that the specific area ratio of the surface layer portion can be used as the initial value.

As shown in FIG. 8B, similar to the degree of change in the specific area ratio of the fatigue portion, the specific area ratio of the fatigue portion has a correlation with the calculated life ratio (fatigue level). Thus, the estimated calculated life ratio can be obtained by using the specific area ratio of the fatigue portion based on the relationship between the specific area ratio of the fatigue portion and the calculated life ratio as a fatigue level database. In this case, the estimated calculated life ratio can be obtained without the initial value.

### Range of Set Value

When obtaining the estimated calculated life ratio using the degree of change in the specific area ratio of the fatigue portion, the set value that is the KAM value for defining the specific area is set within the range of 0 deg to 15 deg.

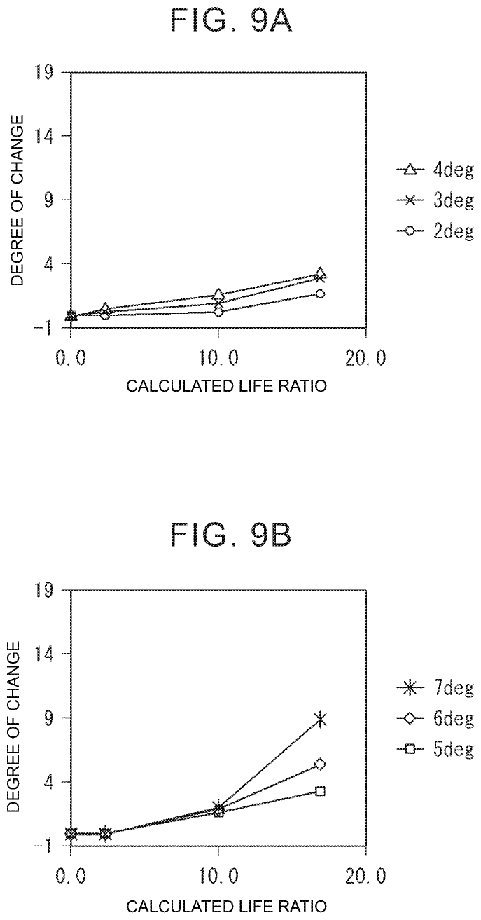

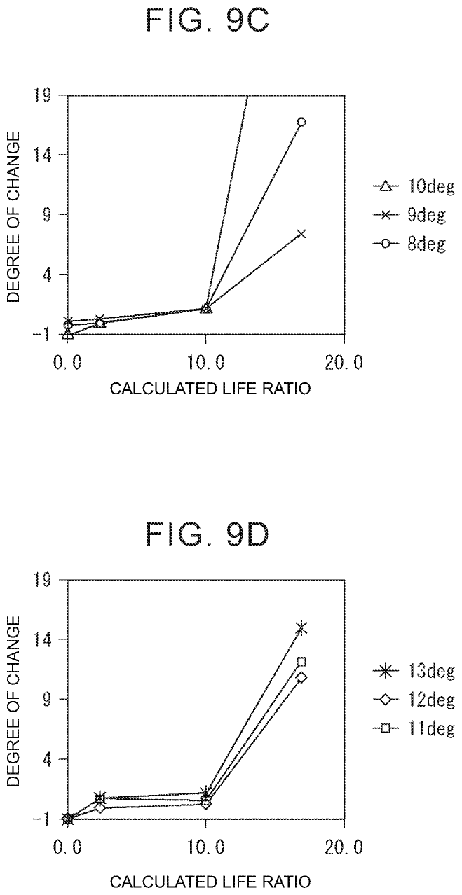

FIG. 9A is a graph of the fatigue level database showing a relationship between the degree of change in the specific area ratio of the fatigue portion and the calculated life ratio when the set value is set in a range of 2 deg to 4 deg. FIG. 9B is a graph of the fatigue level database showing the relationship between the degree of change in the specific area ratio of the fatigue portion and the calculated life ratio when the set value is set in a range of 5 deg to 7 deg. FIG. 9C is a graph of the fatigue level database showing the relationship between the degree of change in the specific area ratio of the fatigue portion and the calculated life ratio when the set value is set in a range of 8 deg to 10 deg. FIG. 9D is a graph of the fatigue level database showing the relationship between the degree of change in the specific area ratio of the fatigue portion and the calculated life ratio when the set value is set in a range of 11 deg to 13 deg.

FIG. 9A shows the fatigue level database when the set values are 2 deg, 3 deg, and 4 deg, and FIG. 9B shows the fatigue level database when the set values are 5 deg, 6 deg, and 7 deg. Further, FIG. 9C shows the fatigue level database when the set values are 8 deg, 9 deg, and 10 deg, and FIG. 9D shows the fatigue level database when the set values are 11 deg, 12 deg, and 13 deg.

These fatigue level databases are obtained by the same method as the above-described creation method for the fatigue level database, and are obtained by using the tapered roller bearings 1. The fatigue level databases are created by plotting the degree of change corresponding to the calculated life ratios of 0, 2.5, 10, and 17.

As shown in FIGS. 9A and 9B, all of the degrees of change in the fatigue level databases with the set values of 2 deg to 7 deg monotonically increase with respect to the calculated life ratio, and it can be understood that there is a good correlation between the degree of change in the specific area ratio of the fatigue portion and the calculated life ratio in all cases.

Further, in FIGS. 9C and 9D, all of the degrees of change in the fatigue level databases with the set values of 8 deg to 13 deg monotonically increase with respect to the calculated life ratio when the calculated life ratio is 10 or more. Thus, the set value can be set within a range of 2 deg to 13 deg as long as the calculated life ratio is 10 or more.

However, as shown in FIG. 9D, in the fatigue level database with the set values of 11 deg to 13 deg, the degree of change may not monotonically increase with respect to the calculated life ratio (as in the case in which the set value is 11 deg) when the calculated life ratio is 10 or less. Further, as shown in FIG. 9C, the fatigue level database with the set value of 10 deg shows an abnormally high value in the degree of change when the calculated life ratio is 17 compared to other cases.

Thus, when obtaining the calculated life ratio of the inner ring 2 based on the degree of change in the specific area ratio of the fatigue portion, the set value is preferably set in the range of 2 deg to 9 deg. This is because a good correlation can be obtained between the degree of change and the calculated life ratio in the above case. When the set value is 10 deg or more, an abnormal value may appear or a range in which the relationship between the degree of change and the calculated life ratio does not show a monotonic increase may appear. Accordingly, the accuracy of the estimated calculated life ratio may be decreased. Therefore, the set value is preferably set in the range of 2 deg to 9 deg.

Further, in this case, the set value is more preferably set in the range of 6 deg to 9 deg. This is because in the range in which the calculated life ratio is 10 or more, an increment of the degree of change with respect to an increment of the calculated life ratio when the set value is set in the range of 6 deg to 9 deg is larger than that in the case of other set values. With the set value in the range of 6 deg to 9 deg, the comparison can be performed with higher accuracy when the calculated life ratios are compared to each other.

Next, the range of the set values when obtaining the calculated life ratio of the inner ring 2 using the specific area ratio of the fatigue portion will be described.

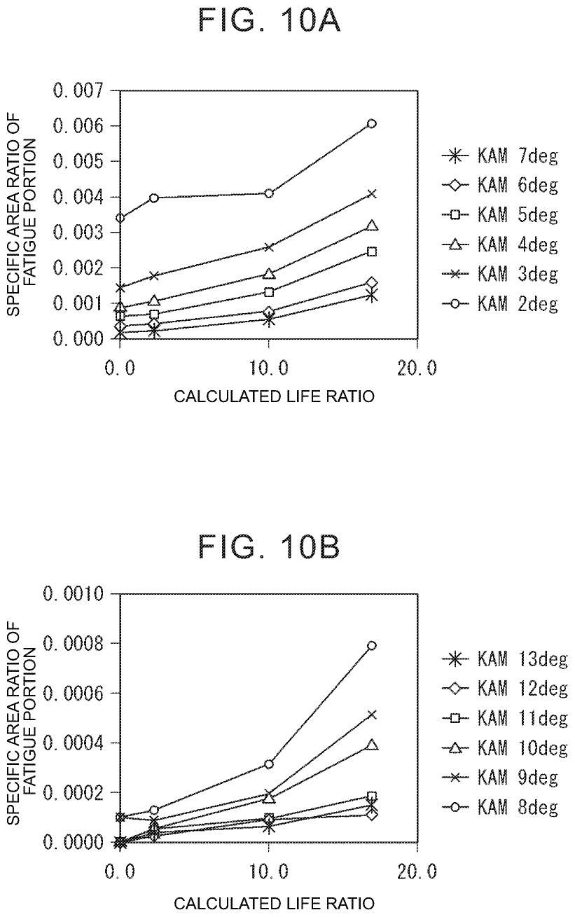

FIG. 10A is a graph of the fatigue level database showing the relationship between the specific area ratio of the fatigue portion and the calculated life ratio when the set value is set in a range of 2 deg to 7 deg, and FIG. 10B is a graph of the fatigue level database showing the relationship between the specific area ratio of the fatigue portion and the calculated life ratio when the set value is set in a range of 8 deg to 13 deg.

FIG. 10A shows the fatigue level database when the set values are 2 deg, 3 deg, 4 deg, 5 deg, 6 deg, and 7 deg. FIG. 10B shows the fatigue level database when the set values are 8 deg, 9 deg, 10 deg, 11 deg, 12 deg, and 13 deg.

As shown in FIG. 10A, all of the specific area ratios of the fatigue portion in the fatigue level database with the set values of 2 deg to 7 deg monotonically increase with respect to the calculated life ratio, and it can be understood that there is a good correlation between the specific area ratio of the fatigue portion and the calculated life ratio in all cases.

Further, in FIG. 10B, all of the specific area ratios of the fatigue portion in the fatigue level database with the set values of 8 deg to 13 deg monotonically increase with respect to the calculated life ratio when the calculated life ratio is 10 or more. Thus, the set value can be set within the range of 2 deg to 13 deg as long as the calculated life ratio is 10 or more.

However, as shown in FIG. 10B, in the fatigue level database with the set values of 9 deg to 13 deg, an increment of the specific area ratio of the fatigue portion with respect to an increment of the calculated life ratio is smaller than that in the case of other set values. Further, in the fatigue level database with the set value of 9 deg, in the range where the calculated life ratio is 10 or less, the specific area ratio of the fatigue portion may not simply increase with respect to the calculated life ratio.

Thus, when obtaining the calculated life ratio of the inner ring 2 based on the specific area ratio of the fatigue portion, the set value is preferably set in a range of 2 deg to 8 deg. This is because a good correlation can be obtained between the specific area ratio of the fatigue portion and the calculated life ratio in the above case. When the set value is 9 deg or more, the increment of the specific area ratio of the fatigue portion with respect to the increment of the calculated life ratio may be small, or a range in which the relationship between the specific area ratio of the fatigue portion and the calculated life ratio does not show a monotonic increase may appear. Accordingly, the accuracy of the estimated calculated life ratio may be decreased. Therefore, the set value is preferably set in the range of 2 deg to 8 deg.

Further, it can be understood from FIG. 10A that the fatigue level database with the set values of 3 deg to 5 deg shows a substantially linear relationship between the specific area ratio of the fatigue portion and the calculated life ratio. Thus, the set value is more preferably set in the range of 3 deg to 5 deg. When the set value is smaller than 3 deg, the relationship between the specific area ratio of the fatigue portion and the calculated life ratio may not always show the linear relationship, and the accuracy of the estimated calculated life ratio that is obtained may be slightly decreased. Similarly, when the set value is larger than 5 deg, the relationship between the specific area ratio of the fatigue portion and the estimated calculated life ratio may not always show the linear relationship, and the accuracy of the estimated calculated life ratio that is obtained may be slightly decreased. By setting the set value within the range of 3 deg to 5 deg, the correlation between the specific area ratio of the fatigue portion and the calculated life ratio can show a good linear relationship.

### Others

The disclosure is not limited to the above embodiment. Misorientation data that can be measured by the EBSD includes, in addition to the KAM value used in the present embodiment, a grain orientation spread (GOS) value, a grain average misorientation (GAM) value, a local average misorientation (LAM) value, and a local orientation spread (LOS) value. Of the above values, the GOS value and the GAM value are called crystal misorientations and are calculated according to a certain rule using the misorientations of all the measurement point areas P in the crystal grains divided by the grain boundary defined earlier in the description to indicate the misorientations with respect to the other crystal grains. Further, the KAM value, the LAM value, and the LOS value are called local misorientations and are calculated according to a certain rule in regions finer than the crystal grains (for example, the regions defined by the measurement point areas P1 to P7 described above) to indicate the misorientations with respect to the other finely divided regions. Here, when the same measurement as in the present embodiment is performed with the LAM value and the LOS value, the same data as in the case of the KAM value can be obtained. Thus, although the above embodiment exemplifies the case in which the estimated calculated life ratio is obtained using the KAM value, the estimated calculated life ratio can be obtained using the LAM value and the LOS value instead of the KAM value. Further, the preferable range of the set value of the specific area using the KAM value is also suitable when using the LAM value or the LOS value.

The above embodiment exemplifies the case in which the calculation device 10 performs the process of obtaining the estimated calculated life ratio based on the degree of change in the existence ratio of the specific area in the fatigue portion in step S5 in FIG. 2. Alternatively, the calculation device 10 may perform the process of obtaining the existence ratio of the specific area in step S4 in FIG. 2 and the process necessary for obtaining the distribution of the KAM values. Alternatively, a computer controlling the SEM and the EBSD may perform the method of the present embodiment.

The above embodiment exemplifies the case in which the specific area ratio of the surface layer portion is used as the initial value that is used in obtaining the degree of change in the specific area ratio of the fatigue portion. Alternatively, the specific area ratio before fatigue has occurred may be used to obtain the degree of change when the specific area ratio before fatigue has occurred is available.

Further, the above embodiment exemplifies the case in which the estimated calculated life ratio is obtained using the degree of change in the specific area ratio of the fatigue portion. Alternatively, as described above, the estimated calculated life ratio may be obtained using the specific area ratio of the fatigue portion, and the estimated calculated life ratio may be obtained using both of the degree of change in the specific area ratio of the fatigue portion and the specific area ratio of the fatigue portion.

Further, the above embodiment exemplifies the case in which the calculated life ratio is used as the fatigue level. Alternatively, for example, upon performing a durability test until damage due to fatigue occurs and regarding a test time from the start of the test to the time when the damage occurs or the like as a standard, the fatigue level may be represented by a ratio with respect to the standard (maximum value).

Further, the above embodiment exemplifies the case in which the fatigue level of the inner ring of the tapered roller bearing is estimated. Alternatively, the fatigue level of an outer ring or rollers can be estimated. The disclosure is not limited to the tapered roller bearing, and the estimated fatigue level of components of other rolling bearings may be obtained. Furthermore, the method according to the disclosure can be applied to mechanical elements in which metal fatigue occurs in addition to rolling bearings. Further, in the above embodiment, the estimated fatigue level is obtained for steel materials such as alloy steel for machine structure and carbon steel for machine structure. Alternatively, the estimated fatigue level of metal materials other than steel materials such as aluminum alloy can be obtained.

What is claimed is:

1. A fatigue level estimation method for estimating a fatigue level of a metal material, the fatigue level estimation method comprising:
estimating a fatigue portion in which fatigue has occurred in the metal material;
measuring a distribution of a misorientation in the fatigue portion;
obtaining a specific area ratio of the fatigue portion based on the distribution of the misorientation in the fatigue portion, the specific area ratio of the fatigue portion being a ratio of a specific area existing in a measurement area of the fatigue portion, and the specific area being an area in which the misorientation is a predetermined set value; and
obtaining an estimated fatigue level of the metal material based on at least one of the specific area ratio of the fatigue portion and a degree of change in the specific area ratio of the fatigue portion.

2. The fatigue level estimation method according to claim 1, further comprising:
measuring a distribution of a misorientation in a surface layer portion between a rolling surface of the metal material and the fatigue portion, the fatigue portion being a portion in which rolling fatigue has occurred and existing in a range in which a depth distance from the rolling surface of the metal material is a predetermined value or more; and
determining a specific area ratio of the surface layer portion based on the distribution of the misorientation in the surface layer portion, the specific area ratio of the surface layer portion being a ratio of the specific area existing in a measurement area of the surface layer portion, wherein obtaining the estimated fatigue level of the metal material is obtaining the degree of change in the specific area ratio of the fatigue portion based on the specific area ratio of the fatigue portion and the specific area ratio of the surface layer portion.

3. The fatigue level estimation method according to claim 1, wherein the misorientation is one of a kernel average misorientation (KAM) value, a local average misorientation (LAM) value, and a local orientation spread (LOS) value.

4. The fatigue level estimation method according to claim 3, wherein in obtaining the estimated fatigue level of the metal material, when the estimated fatigue level of the metal material is obtained based on the specific area ratio of the fatigue portion, the predetermined set value is set within a range of 2 degrees to 8 degrees.

5. The fatigue level estimation method according to claim 4, wherein the predetermined set value is set within a range of 3 degrees to 5 degrees.

6. The fatigue level estimation method according to claim 3, wherein in obtaining the estimated fatigue level of the metal material, when the estimated fatigue level of the metal material is obtained based on the degree of change in the specific area ratio of the fatigue portion, the predetermined set value is set within a range of 2 degrees to 9 degrees.

7. The fatigue level estimation method according to claim 1, further comprising creating a database showing a relationship between the fatigue level of the metal material and at least one of the specific area ratio of the fatigue portion and the degree of change in the specific area ratio of the fatigue portion, wherein in obtaining the estimated fatigue level of the metal material, the estimated fatigue level of the metal material is obtained by referring to the database.

8. A creation method for a database for a fatigue level estimation for creating the database for the fatigue level estimation of a metal material, the database being used in obtaining the estimated fatigue level of the metal material based on at least one of the specific area ratio of the fatigue portion and the degree of change in the specific area ratio of the fatigue portion in the fatigue level estimation method according to claim 1, the creation method for the database comprising:
obtaining a plurality of test samples made of the same material as the metal material, each of the test samples having a different fatigue level;
estimating the fatigue portion in which fatigue has occurred in each of the test samples;
measuring a distribution of a misorientation in the fatigue portion in each of the test samples;
obtaining the specific area ratio of the fatigue portion for each of the test samples based on the distribution of the misorientation in the fatigue portion; and
associating the fatigue level of each of the test samples with at least one of the specific area ratio of the fatigue portion of each of the test samples and the degree of change in the specific area ratio of the fatigue portion of each of the test samples to create the database for the fatigue level estimation.
